(19) United States
(12) Patent Application Publication
(10) Pub. No.: US 2009/0222080 A1
(43) Pub. Date: Sep. 3, 2009

(54) MEDICAL STENT PROVIDED WITH INHIBITORS OF TUMOR NECROSIS FACTOR-ALPHA

(75) Inventors: Johan Wouter Jukema, Oegstgeest (NL); Paulus Hubertus Andreas Quax, Voorschoten (NL); Ronald Andrianus Maria Horvers, Geldrop (NL)

Correspondence Address: KNOBBE MARTENS OLSON & BEAR LLP, 2040 MAIN STREET, FOURTEENTH FLOOR, IRVINE, CA, 92614, US

(73) Assignee: Picarus NV SA, Luxembourg (LU)

(21) Appl. No.: 12/093,095

(22) PCT Filed: Nov. 8, 2005
(86) PCT No.: PCT/EP05/11943

§ 371 Date: Aug. 14, 2008

(51) Int. Cl. A61F 2/06

(52) U.S. Cl. 623/1.42; 623/1.15

(57) ABSTRACT

A stent provided with a composition that includes at least one inhibitor of TNF-alpha is disclosed. The stent is useful in treating smooth muscle cell proliferation, such as stenosis and preventing restenosis in vascular ducts.

### FIELD OF THE INVENTION

The present invention relates to a stent useful for expanding a duct lumen of a subject and treating restenosis therein.

### BACKGROUND TO THE INVENTION

A stent is commonly used as a tubular structure introduced inside the lumen of a duct to relieve an obstruction. Commonly, stents are inserted into the lumen of the duct in a non-expanded form and are then expanded autonomously (or with the aid of a second device) in situ.

When a stent is used to expand a vascular lumen, restenosis (re-narrowing) may occur. Restenosis of an artherosclerotic coronary artery after a stand-alone angioplasty may occur in 10-50% of patients within 6 months, requiring either further angioplasty or coronary artery bypass graft. It is presently understood that the process of fitting a bare stent (without any drug), besides opening the artherosclerotically obstructed artery, also injures resident coronary arterial smooth muscle cells (SMC). In response to this trauma, adhering platelets, infiltrating macrophages, leukocytes, or the smooth muscle cells (SMC) themselves release cell derived growth factors with subsequent proliferation and migration of medial SMC through the internal elastic lamina to the area of the vessel intima. Further proliferation and hyperplasia of intimal SMC and, most significantly, production of large amounts of extracellular matrix over a period of 3-6 months results in the filling in and narrowing of the vascular space sufficient to significantly obstruct coronary blood flow.

To reduce or prevent restenosis, stents are provided with a means for delivering an inhibitor of SMC proliferation directly to the wall of the expanded vessel. Such delivery means include, for example, via the struts of a stent, a stent graft, grafts, stent cover or sheath, composition with polymers (both degradable and nondegrading) to hold the drug to the stent or graft or entrapping the drug into the metal of the stent or graft body which has been modified to contain micropores or channels. Other delivery means include covalent binding of the drug to the stent via solution chemistry techniques (such as via the Carmeda process) or dry chemistry techniques (e.g. vapour deposition methods such as rf-plasma polymerization) and combinations thereof. Examples of some means for delivery are mentioned in patent document U.S. Pat. No. 6,599,314.

Inhibitors of SMC proliferation include sirolimus (or rapamycin, an immunosuppressive agent) and paclitaxel (or taxol, an antiproliferative, anti-angiogenic agent). Other agents which have demonstrated the ability to reduce myointimal thickening in animal models of balloon vascular injury are heparin, angiopeptin (a somatostatin analog), calcium channel blockers, angiotensin converting enzyme inhibitors (captopril, cilazapril), cyclosporin A, trapidil (an antianginal, antiplatelet agent), terbinafine (antifungal), colchicine (antitubulin antiproliferative), and c-myc and c-myb antisense oligonucleotides.

The problem with inhibitors of the art is the delayed healing (Farb A, et al. Circulation 2001;104:473-479) and polymer-related hypertensitivity reactions. (Virmani R et al. Circulation 2004;109:r8-r42.) This increases the risk of delayed, potentially fatal thrombosis. (Virmani R et al., Liistro F, Colombo A. Heart 2001;86:262-264.)

In view of the prior art, there is a need for a new type of inhibitor of stenosis and restenosis, delivered via a stent.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “a stent” means one stent or more than one stent.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of stents, and can also include 1.5, 2, 2.75 and 3.80, when referring to, for example, doses).

The present invention relates to a stent provided with a composition comprising at least one inhibitor tumour necrosis factor-alpha (TNF-alpha) for use in treating SMC proliferation in vascular ducts. Where a particular use of a composition of the present invention is described, said use may be understood as a method.

The composition can be used for treating SMC proliferation. This means that the composition can be used to treat a stenosing or restenosing cell mass. The mass may be shrunk or completely eradicated by the composition. It also means the composition can prevent restenosis when applied to regions from which stenosing or restenosing cells have been surgically removed, to reduce the possibility of regrowth. The stent allows treatment of MSC proliferation over a prolonged period.

The inventors have found that mice models having TNF-alpha polymorphisms which decrease the production of active TNF-alpha, have a decreased risk of SMC proliferation. Therefore, administration of inhibitors of TNF-alpha, such as, for example, thalidomide, to the stenosing or restenosing cells provides an effective treatment against such proliferating cells. This has been shown to be the case. Using TNF-alpha inhibitors reduces or arrests SMC proliferation early in the cell cycle leading to a better healing response and a less aggressive treatment where other cells are killed. Inhibiting TNF-alpha leads to cytostatis as it induces growth arrest of cells in the G₁ phase of the cell cycle. Furthermore, the inhibition of TNF-alpha decreases the release of inflammatory cytokines and reduces cell adhesion molecule expression. These processes aid in the prevention of restenosis.

The term “duct” as used herein refers to any walled cavity of a subject suitable for placing a medical stent therein. Such a duct may be narrowed by a medical condition such as stenosis or atherosclerosis. Examples of ducts include, but are not limited to arteries and veins.

A “subject” according to the present invention may be any living body susceptible to treatment by a stent. Examples include, but are not limited to humans, dogs, cats, horses, cows, sheep, rabbits, and goats etc.

Where a stent is provided with a composition, it means the composition is deposited on, or within the stent, so the composition can released when the stent contacts the duct. The stent may be coated with the composition, Alternatively, the stent may be impregnated with composition, Alternatively, the stent may comprise cavities in which the composition resides. Various embodiments of the stent are described below.

A composition as used herein may comprise at least TNF-alpha inhibitor. In the preferred mode of the invention, a stent is provided a TNF-alpha inhibitor which is thalidomide.

A composition of the invention may comprise additional substances, such as, for example, those that facilitate dissolving the inhibitor and/or the attachment of the inhibitor to the stent, those that release the inhibitor in a controlled manner in situ, and those that facilitate the functioning or the performance of the stent in situ. Such additional substances are known to the skilled artisan.

Stents

Stents according to the invention may be any stent that is capable of being provided with a composition according to the invention. Stents have been extensively described in the art. For example they may be cylinders which are perforated with passages that are slots, ovoid, circular, regular, irregular or the like shape. They may also be composed of helically wound or serpentine wire structures in which the spaces between the wires form the passages. Stents may also be flat perforated structures that are subsequently rolled to form tubular structures or cylindrical structures that are woven, wrapped, drilled, etched or cut to form passages. A stent may also be combined with a graft to form a composite medical device, often referred to as a stent graft. A stent should capable of being coated with a composition described herein.

Stents may be made of biocompatible materials including biostable and bioabsorbable materials. Suitable biocompatible metals include, but are not limited to, stainless steel, tantalum, titanium alloys (including nitinol), and cobalt alloys (including cobalt-chromium-nickel alloys). Stents may be made of biocompatible and bioabsorbable materials such as magnesium based alloys. Bioabsorbable stents may inserted at the site of treatment, and left in place. The structure of the stent does not become incorporated into the wall of the duct being treated, but is degraded with time. Where the stent is made from biostable (non-absorbable) materials, the stent may be inserted for the duration of treatment and later removed.

Suitable nonmetallic biocompatible materials include, but are not limited to, polyamides, polyolefins (i.e. polypropylene, polyethylene etc.), nonabsorbable polyesters (i.e. polyethylene terephthalate), and bioabsorbable aliphatic polyesters (i.e. homopolymers and copolymers of lactic acid, glycolic acid, lactide, glycolide, para-dioxanone, trimethylene carbonate, epsilon-caprolactone, etc. and blends thereof), lactide capronolactone, poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), poly(hydroxy butyrate), polyacrylate, polyacrylamid, poly(hydroxyethyl methacrylate), elastin polypeptide co-polymer, polyurethane, starch, polysiloxane and their copolymers.

Stents according to the present invention can be of any type known in the art suitable for delivery of TNF-alpha inhibitors. As such these stents can be balloon expandable, self-expanding, provided with cavities etched into the framework of the stent for containing substances, stents provided with means for containing substances, bioabsorbable stents. The stent may also be made from different sorts of wires, for instance from polymeric biodegradable wires containing the active compound, interweaved with the metallic struts of the stent (balloon expendable or self-expandable stent).

Self expanding stents may be braided, from flexible metal, such as special alloys, from nitenol, from phynox. Self-expandable stents made from nitenol may be laser cut. One or more of the filaments that compose the self-expandable stent can be made from a polymer or a tube that elutes the anti-energetic compound.

Variations of stent and polymers are described in more detailed below.

Examples of stents include, but are not limited to, those described in U.S. Pat. No. 4,733,665, U.S. Pat. No. 4,800,882, U.S. Pat. No. 4,886,062, U.S. Pat. No. 5,514,154, U.S. Pat. No. 6,398,806, EP 1 140 242, U.S. Pat. No. 6,248,129, EP 1 217 969, EP 1 359 868, EP 1 349 517, EP 1 347 717, EP 1 318 765, EP 1 296 615, EP 1 229 864, EP 1 194 081, EP 1 191 904, EP 1 139 914, EP 1 087 701, EP 1 079 768, EP 1 018 985, EP 0 749 729, EP 0 556 850, EP 1 328 212, EP 1 322 256, EP 0 740 558, EP 1 251 800, EP 1 251 799, EP 1 235 856, EP 1 227 772, EP 1 123 065, EP 1 112 040, EP 1 094 764, EP 1 076 534, EP 1 065 993, EP 1 059 896, EP 1 059 894, EP 1 049 421, EP 1 027 012, EP 1 001 718, EP 0 986 416, EP 0 859 644, EP 0 740 558, EP 0 664 689, EP 0 556 850, EP 1 372 535, U.S. Pat. No. 6,669,723, U.S. Pat. No. 6,663,660, EP 1 065 996, U.S. Pat. No. 6,652,577, U.S. Pat. No. 6,652,575, EP 1 360 943, U.S. Pat. No. 6,620,202, U.S. Pat. No. 6,610,087, U.S. Pat. No. 6,602,283, U.S. Pat. No. 6,592,617, U.S. Pat. No. 6,585,758, U.S. Pat. No. 6,585,753, EP 1 318 771, EP 1 318 768, U.S. Pat. No. 6,579,308, US 2003/0109931, EP 1 163 889, EP0 790 811, U.S. Pat. No. 6,551,351, U.S. Pat. No. 6,540,777, U.S. Pat. No. 6,533,810, U.S. Pat. No. 6,530,950, U.S. Pat. No. 6,527,802, U.S. Pat. No. 6,524,334, U.S. Pat. No. 6,506,211, U.S. Pat. No. 6,488,703, U.S. Pat. No. 6,485,590, U.S. Pat. No. 6,478,816, U.S. Pat. No. 6,478,815, U.S. Pat. No. 6,475,233, EP 1 251 891, U.S. Pat. No. 6,471,720, U.S. Pat. No. 6,428,569, EP 1 223 873, EP 0 758 216, U.S. Pat. No. 6,416,543, U.S. Pat. No. 6,641,538, EP 1 217 101, U.S. Pat. No. 6,409,754, U.S. Pat. No. 6,409,753, US 2002/0077592, EP 0 754 017, U.S. Pat. No. 6,398,807, EP 1 207 815, EP 0 775 471, U.S. Pat. No. 6,395,020, IS 6,391,052, U.S. Pat. No. 6,387,122, U.S. Pat. No. 6,379,379, U.S. Pat. No. 6,355,070, U.S. Pat. No. 6,348,065, EP1 173 110, U.S. Pat. No. 6,334,870, EP1 163 889, IS 6,325,822, U.S. Pat. No. 6,319,277, EP 1 144 042, EP0 622 059, U.S. Pat. No. 6,261,319, EP1 112 039, U.S. Pat. No. 6,251,134, U.S. Pat. No. 6,240,978, U.S. Pat. No. 6,217,607, U.S. Pat. No. 6,193,744, U.S. Pat. No. 6,174,328, EP 1 065 996, U.S. Pat. No. 6,168,621, U.S. Pat. No. 6,168,619, U.S. Pat. No. 6,159,238, U.S. Pat. No. 6,159,237, U.S. Pat. No. 6,146,416, U.S. Pat. No. 6,143,002, EP 0 986 416, EP 1 032 329, EP 1 019 107, EP 1 011 529, EP 1 011 528, U.S. Pat. No. 6,071,308, EP0 859 644, U.S. Pat. No. 6,059,810, U.S. Pat. No. 6,042,597, U.S. Pat. No. 6,033,433, EP 0 979 059, U.S. Pat. No. 6,022,371, U.S. Pat. No. 5,993,483, U.S. Pat. No. 6,957,974, U.S. Pat. No. 5,594,744, WO 99/44535, EP 0 934 034, EP 0 934 033, U.S. Pat. No. 5,922,019, WO 99/16388, EP 0 858 298, U.S. Pat. No. 5,891,191, U.S. Pat. No. 5,888,201, U.S. Pat. No. 5,876,448, U.S. Pat. No. 5,876,445, U.S. Pat. No. 5,868,781, U.S. Pat. No. 5,855,600, WO 98/55174, EP 0 746 375, U.S. Pat. No. 5,824,045, U.S. Pat. No. 5,800,511, EP 0 836 839, EP 0 767 685, U.S. Pat. No. 5,683,488, EP 1 212 987, EP 1 212 987, EP1 151 730, EP1 151 730, EP 0 722 701, EP 1 236 447, EP 1 293 178, EP 1 236 449, EP1 190 685, EP1 138 280, EP 1 346 706, EP 1 330 993, EP 1 258 231, EP 1 325 717, EP 1 302 179, EP 1 095 634, EP 1 302 179, EP 1 295 615, EP 1 293 178, EP 1 266 638, EP 1 266 638, EP 1 260 197, EP 1 236 448, EP 1 236 446, EP 1 236 445, EP 1 258 231, EP 1 236 449, EP 1 236 448, EP 1 236 447, EP 1 236 446, EP 1 236 445, EP1 112 724, EP1 190 685, EP1 179 323, EP 1 138 280,EP1 112 724,EP1 031 330,EP0 937 442,EP1 042 997,EP1 031 330,EP 1 025 812, EP 1 000 590, EP 0 950 386, EP 0 873 734, EP 0 937 442, EP 0 864 302, EP 0 928 606, EP 0 864 302, EP 0 806 191, EP 1 212 988, EP 1 266 640, EP 1 266 639, EP 0 879 027, EP 1 226 798, U.S. Pat. No. 6,656,215, EP 1 362 564, EP 0 904 009, EP 1 212 990, EP 1 155 664, EP1 121 911, EP 1 266 640, EP 1 266 639, EP 1 031 329, EP 1 212 990, EP 1 212 988, EP0 698 380, EP1 155 664, EP1 121 911, U.S. Pat. No. 6,270,521, EP 1 031 329, EP 0 928 605, EP 0 928 605, EP 0 904 009, EP 0 903 123, EP 0 879 027, U.S. Pat. No. 5,840,009, EP 0 839 506, U.S. Pat. No. 5,741,324, EP0 823 245, EP0 698 380, EP0 606 165, U.S. Pat. No. 6,626,938, U.S. Pat. No. 6,613,075, U.S. Pat. No. 6,565,600, U.S. Pat. No. 6,562,067, U.S. Pat. No. 6,547,817, U.S. Pat. No. 6,540,775, U.S. Pat. No. 6,520,985, U.S. Pat. No. 6,494,908, U.S. Pat. No. 6,482,227, U.S. Pat. No. 6,423,091, U.S. Pat. No. 6,342,067, U.S. Pat. No. 6,325,825, U.S. Pat. No. 6,315,708, U.S. Pat. No. 6,267,783, U.S. Pat. No. 6,267,777, U.S. Pat. No. 6,264,687, U.S. Pat. No. 6,258,116, U.S. Pat. No. 6,238,409, U.S. Pat. No. 6,214,036, U.S. Pat. No. 6,190,406, U.S. Pat. No. 6,176,872, U.S. Pat. No. 6,162,243, U.S. Pat. No. 6,129,755, U.S. Pat. No. 6,053,873, EP 0 904 009, U.S. Pat. No. 6,019,778, EP 0 974 315, U.S. Pat. No. 6,017,363, EP 0 951 877, EP 0 970 711, EP 0 785 807, U.S. Pat. No. 6,013,019, EP 0 947 180, U.S. Pat. No. 5,997,570, U.S. Pat. No. 5,980,553, EP 0 951 877, U.S. Pat. No. 5,938,682, U.S. Pat. No. 5,895,406, U.S. Pat. No. 5,891,108, U.S. Pat. No. 5,882,335, U.S. Pat. No. 5,792,172, U.S. Pat. No. 5,782,906, EP 0 810 845, U.S. Pat. No. 5,695,516, U.S. Pat. No. 5,674,241, U.S. Pat. No. 5,609,605, U.S. Pat. No. 5,607,442, U.S. Pat. No. 5,599,291, U.S. Pat. No. 5,135,536, U.S. Pat. No. 5,116,365, EP 0 378 151, U.S. Pat. No. 4,994,071, EP 0 378 151, U.S. Pat. No. 4,856,516. The content of all documents referred to in this application are incorporated herein by reference.

Polymers

It is an aspect of the invention that the stent is provided with at least one inhibitor of TNF-alpa by way of at least partially coating the stent with a composition comprising a polymer. A polymer according to the present invention is any that facilitates attachment of the inhibitor(s) to the stent (i.e. stent and/or membrane) and/or facilitates the controlled release of said inhibitors.

Polymers suitable for use in the present invention are any that are capable of attaching to the stent and releasing inhibitor. They must be biocompatible to minimize irritation to the duct wall. Polymers may be, for example, film-forming polymers that are absorbable or non-absorbable. The polymer may be biostable or bioabsorbable depending on the desired rate of release or the desired degree of polymer stability.

Suitable bioabsorbable polymers that could be used include polymers selected from the group consisting of aliphatic polyesters, poly(amino acids), copoly(ether-esters), polyalkylenes oxalates, polyamides, poly(iminocarbonates), polyanhydrides, polyorthoesters, polyoxaesters, polyamidoesters, polylactic acid (PLA), polyethylene oxide (PEO), polycaprolactone (PCL), polyhydroxybutyrate valerates, polyoxaesters containing amido groups, poly(anhydrides), polyphosphazenes, silicones, hydrogels, biomolecules and blends thereof.

For the purpose of the present invention, aliphatic polyesters include homopolymers and copolymers of lactide (which includes lactic acid D-, L- and meso lactide), epsilon-caprolactone, glycolide (including glycolic acid), hydroxybutyrate, hydroxyvalerate, paradioxanone, trimethylene carbonate (and its alkyl derivatives), 1,4-dioxepan-2-one, 1,5-dioxepan-2-one, 6,6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30(1), page 498, 1989 (e.g. PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein).

Polyphosphazenes, co-, ter- and higher order mixed monomer based polymers made from L-lactide, D,L-lactide, lactic acid, glycolide, glycolic acid, para-dioxanone, trimethylene carbonate and epsilon-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein).

Polyanhydrides from diacids of the form HOOC—C₆H₄—O—(CH₂)m-O—C₆H₄—COOH wherein m is an integer in the range of from 1 to 11, 3 to 9, 3 to 7, 2 to 6 or preferably 2 to 8, and copolymers thereof with aliphatic alpha-omega diacids of up to 8, 9, 10, 11 or preferably 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference).

Other polymeric biomolecules for the purpose of this invention include naturally occurring materials that may be enzymatically degraded in the human body or are hydrolytically unstable in the human body such as fibrin, fibrinogen, collagen, gelatin, glycosaminoglycans, elastin, and absorbable biocompatible polysaccharides such as chitosan, starch, fatty acids (and esters thereof), glucoso-glycans and hyaluronic acid.

Suitable biostable polymers with relatively low chronic tissue response, such as polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters could also be used. Other polymers could also be used if they can be dissolved, cured or polymerized on the stent. These include polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile, polyvinyl ketones; polyvinyl aromatics such as polystyrene; polyvinyl esters such as polyvinyl acetate; copolymers of vinyl monomers with each other and olefins, such as etheylene-methyl methacrylate copolymers, acrylonitrile-styrene copolymers, ABS resins and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e. carboxymethyl cellulose and hydoxyalkyl celluloses); and combinations thereof. Polyamides for the purpose of this application would also include polyamides of the form—NH—(CH₂)n-CO— and NH—(CH₂)x-NH—CO—(CH₂)y-CO, wherein

n is an integer in from 5 to 15, 7 to 11, 8 to 10 or preferably 6 to 13;

x is an integer in the range of from 5 to 14, 7 to 11, 8 to 10 or preferably 6 to 12; and

y is an integer in the range of from 3 to 18, 5 to 14, 6 to 10 or preferably 4 to 16. The list provided above is illustrative but not limiting.

Other polymers suitable for use in the present invention are bioabsorbable elastomers, more preferably aliphatic polyester elastomers. In the proper proportions aliphatic polyester copolymers are elastomers. Elastomers present the advantage that they tend to adhere well to the metal stents and can withstand significant deformation without cracking. The high elongation and good adhesion provide superior performance to other polymer coatings when the coated stent is expanded. Examples of suitable bioabsorbable elastomers are described in U.S. Pat. No. 5,468,253 hereby incorporated by reference. Preferably the bioabsorbable biocompatible elastomers based on aliphatic polyester, including but not limited to those selected from the group consisting of elastomeric copolymers of epsilon-caprolactone and glycolide (preferably having a mole ratio of epsilon-caprolactone to glycolide of from about 35:65 to about 65:35, more preferably 45:55 to 35:65) elastomeric copolymers of E-caprolactone and lactide, including L-lactide, D-lactide blends thereof or lactic acid copolymers (preferably having a mole ratio of epsilon-caprolactone to lactide of from about 35:65 to about 90:10 and more preferably from about 35:65 to about 65:35 and most preferably from about 45:55 to 30:70 or from about 90:10 to about 80:20) elastomeric copolymers of p-dioxanone (1,4-dioxan-2-one) and lactide including L-lactide, D-lactide and lactic acid (preferably having a mole ratio of p-dioxanone to lactide of from about 30:70 to about 70:30, 45:55 to about 55:45, and preferably from about 40:60 to about 60:40) elastomeric copolymers of epsilon-caprolactone and p-dioxanone (preferably having a mole ratio of epsilon-caprolactone to p-dioxanone of from about 40:60 to about 60:40 and preferably from about 30:70 to about 70:30) elastomeric copolymers of p-dioxanone and trimethylene carbonate (preferably having a mole ratio of p-dioxanone to trimethylene carbonate of from about 40:60 to about 60:40, and preferably from about 30:70 to about 70:30), elastomeric copolymers of trimethylene carbonate and glycolide (preferably having a mole ratio of trimethylene carbonate to glycolide of from about 40:60 to about 60:40 and preferably from about 30:70 to about 70:30), elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. As is well known in the art these aliphatic polyester copolymers have different hydrolysis rates, therefore, the choice of elastomer may in part be based on the requirements for the coatings adsorption. For example epsilon-caprolactone-co-glycolide copolymer (45:55 mole percent, respectively) films lose 90% of their initial strength after 2 weeks in simulated physiological buffer whereas the epsilon-caprolactone-co-lactide copolymers (40:60 mole percent, respectively) loses all of its strength between 12 and 16 weeks in the same buffer. Mixtures of the fast hydrolyzing and slow hydrolyzing polymers can be used to adjust the time of strength retention.

The amount of coating may range from about 0.5 to about 20 as a percent of the total weight of the stent after coating and preferably will range from about 1 to about 15 percent. The polymer coatings may be applied in one or more coating steps depending on the amount of polymer to be applied. Different polymers may also be used for different layers in the stent coating. In fact it may be an option to use a dilute first coating solution as primer to promote adhesion of a subsequent coating layers that may contain inhibitor.

Additionally, a top coating can be applied to further delay release of the inhibitory pharmaceutical agent, or they could be used as the matrix for the delivery of a different pharmaceutically active material. The amount of top coatings on the stent may vary, but will generally be less than about 2000 micrograms, preferably the amount of top coating will be in the range of about micrograms to about 1700 micrograms and most preferably in the range of from about 300 micrograms to 1000 about micrograms. Layering of coating of fast and slow hydrolyzing copolymers can be used to stage release of the drug or to control release of different agents placed in different layers. Polymer blends may also be used to control the release rate of different agents or to provide desirable balance of coating (i.e. elasticity, toughness etc.) and drug delivery characteristics (release profile). Polymers with different solubilities in solvents can be used to build up different polymer layers that may be used to deliver different drugs or control the release profile of a drug. For example since epsilon-caprolactone-co-lactide elastomers are soluble in ethyl acetate and epsilon-caprolactone-co-glycolide elastomers are not soluble in ethyl acetate. A first layer of epsilon-caprolactone-co-glycolide elastomer containing a drug can be over coated with epsilon-caprolactone-co-glycolide elastomer using a coating solution made with ethyl acetate as the solvent. Additionally, different monomer ratios within a copolymer, polymer structure or molecular weights may result in different solubilities. For example, 45/55 epsilon-caprolactone-co-glycolide at room temperature is soluble in acetone whereas a similar molecular weight copolymer of 35/65 epsilon-caprolactone-co-glycolide is substantially insoluble within a 4 weight percent solution. The second coating (or multiple additional coatings) can be used as a top coating to delay the drug delivery of the drug contained in the first layer. Alternatively, the second layer could contain a different inhibitor to provide for sequential inhibitor delivery. Multiple layers of different inhibitors could be provided by alternating layers of first one polymer then the other. As will be readily appreciated by those skilled in the art numerous layering approaches can be used to provide the desired drug delivery.

The coatings can be applied by suitable methodology known to the skilled person, such as, for example, dip coating, spray coating, electrostatic coating, melting a powered form onto the stent. The coating may also be applied during the intervention by the interventional cardiologist on a bare stent. As some polymers (for instance polyorthoesters) need special conservation conditions (argon atmosphere and cold temperature), the drug with the coating may be delivered in a special packing. The MD would apply the coating on the bare stent surface—as it is sligthly sticky—just before introducing the premounted stent inside the patient duct or cavity.

Other examples of polymeric coatings, and coating methods are given in patent documents EP 1 107 707, WO 97/10011, U.S. Pat. No. 6,656,156, EP 0 822 788, U.S. Pat. No. 6,364,903, U.S. Pat. No. 6,231,600, U.S. Pat. No. 5,837,313, WO 96/32907, EP 0 832,655, U.S. Pat. No. 6,653,426, U.S. Pat. No. 6,569,195, EP 0 822 788 B1, WO 00/32238, U.S. Pat. No. 6,258,121, EP 0 832,665, WO 01/37892, U.S. Pat. No. 6,585,764, U.S. Pat. No. 6,153,252 which are incorporated herein by reference.

Non-polymeric Coatings

Another aspect of the invention is a stent coated with a composition of the invention, wherein the presence of a polymer is optional. Such stents suited to polymeric and non-polymeric coatings and compositions are known in the art. These stents may, for example, have a rough surface, microscopic pits or be constructed from a porous material. Examples include, but are not limited to the disclosures of U.S. Pat. No. 6,387,121, U.S. Pat. No. 5,972,027, U.S. Pat. No. 6,273,913 and U.S. Pat. No. 6,099,561. These documents are incorporated herein by reference.

Stent Grafts

A stent may also be combined with a graft to form a composite medical device, often referred to as a stent graft. Such a composite medical device provides additional support for blood flow through weakened sections of a blood vessel. The graft element made be formed from any suitable material such as, for example, textiles such as nylon, Orlon, Dacron, or woven Teflon, and nontextiles such as expanded polytetrafluroethylene (ePTFE).

Stent grafts of the present invention may be coated with, or otherwise adapted to release the TNF-alpha inhibitor(s) of the present invention. Stent grafts may be adapted to release such inhibitors by (a) directly affixing to the stent graft a composition according to the invention (e.g., by either spraying the stent graft with a polymer/inhibitor film, or by dipping the implant or device into a polymer/drug solution, or by other covalent or noncovalent means); (b) by coating the stent graft with a substance such as a hydrogel which will in turn absorb a composition according to the invention; (c) by interweaving a composition coated thread into the stent graft (e.g., a polymer which releases the inhibitor formed into a thread into the implant or device; (d) by inserting a sleeve or mesh which is comprised of or coated with a composition according to the present invention; (e) constructing the stent graft itself a composition according to the invention; or (f) otherwise impregnating the stent graft with a composition according to the invention.

The stent graft may be biodegradable, made from, but not limited to, magnesium alloy and starch.

Examples and methods of stent graft coating are provided in patent documents WO 00/40278, and WO 00/56247. These documents are incorporated herein by reference.

Stent Cavities

It is an aspect of the invention that the stent is provided with a composition of the invention which is present in a cavity formed in the stent. Stent in which cavities are present suitable for the delivery of biologically active material are known in the art, for example, from WO 02/060351 U.S. Pat. No. 6,071,305, U.S. Pat. No. 5,891,108. These documents are incorporated herein by reference.

Biodegradable Stents

Another aspect of the invention is a biodegradable (bioabsorbable) stent impregnated with a composition according to the present invention. The composition may be coated onto the stent or impregnated into the stent structure, said composition released in situ concomitant with the biodegradation of the stent. Suitable materials for the main body of the stent includes, but are not limited to poly(alpha-hydroxy acid) such as poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen or other connective proteins or natural materials, poly(hydroxybutyrate), polyanhydride, polyphosphoester, poly(amino acids), hylauric acid, starch, chitosan, adhesive proteins, co-polymers of these materials as well as composites and combinations thereof and combinations of other biodegradable polymers. Biodegradable glass or bioactive glass is also a suitable biodegradable material for use in the present invention. A composition of the present invention may be incorporated into a biodegradable stent using known methods. Examples of biodegradable stents known in the art, include, but are not limited to the those disclosed in US 2002/0099434, U.S. Pat. No. 6,387,124 B1, U.S. Pat. No. 5,769,883, EP 0 894 505 A2, U.S. Pat. No. 653,312, U.S. Pat. No. 6,423,092, U.S. Pat. No. 6,338,739 and U.S. Pat. No. 6,245,103, EP 1 110 561. These documents are incorporated here by reference. Biodegradable stents may also be made from a metal (lanthanide such as, but not limited to magnesium or magnesium alloy), or an association of organic and non-organic material (such as, but not limited to a magnesium based alloy combined with starch).

Medical Treatments

As mentioned above, SMC proliferation such as stenosis, resteonsis and its prevention are susceptible to treatment by a stent according to the present invention. A stent may be placed on or adjacent to the proliferating SMCs, for example, in a duct such as an artery. The stent may also be placed in situ after the removal of proliferating SMCs. For example, after surgical removal of a stenosis in an artery, a stent may be placed in the area of the artery suture to shrink proliferating cells possibly remaining after surgery.

The inhibitor may be combined with a slow release agent so that the inhibitor can act over a period of days to weeks, so avoiding replacement of the stent. Where a biodregradable stent is used, the stent does not need to be removed after treatment.

The present invention is useful for treating any animal in need including humans, livestock, domestic animals, wild animals, or any animal in need of treatment. Examples of an animal is human, horse, cat, dog, mice, rat, gerbil, bovine species, pig, fowl, camelidae species, goat, sheep, rabbit, hare, bird, elephant, monkey, chimpanzee etc. An animal may be a mammal.

TNF-alpha Inhibitors

The TNF-alpha inhibitors of the present of the invention may be any inhibitor that at least partially inactivate TNF alpha i.e. they prevent TNF-alpha from binding to the natural target such as TNF-alpha receptor. They also include inhibitors which at least partially inactive biosynthesis of TNF alpha.

Inhibitors of TNF-alpha are any known in the art. Examples of TNF-alpha inhibitors include thalidomide also known as alpha-(N-phthalimido) glutarimide or 2-(2,6-dioxo-3-piperidinyl)-1H-isoindole-1,3(2H)-dione). Preferably, thalidomide is is the R(+) enantiomer of thalidomide. Other inhibitors of TNF-alpha include pentoxifylline, RWJ67657, EtOH (alcohol), Tyrphostin AG-556, AG126, AG128, s-adenosylmethionine, 5′-methylthioadenosine, inliximab (remicade), tgAAC94, entanercept (enbrel), D2E7(humira), MP inhibitor GI5402, TMI-1, SCIO-469, VX-745, BIRB 796, BB-2275, Uncaria tomentosa, SteiHap69, TACE inhibitors, ISIS25302, roflumilast, AS2868, AS2920, SPC-839, PEG-TNFRI, CDP-870, onercept, TIMP-3, DPC333, MMP inhibitors, Bortezomib (velcade), oxaspirodion, Shi-Bi-Lin, JM34, luteolin, urtica dioica, green tea polyphenols, tenidap, leflunimide (arava), curcuminoids, dexamethasone, vitamin D3, prostaglandin E2, cyclosporin A. TNF-alpha inhibitors includes derivatives or salts of the native TNF-alpha inhibitor.

Inhibitors of TNF-alpha may be based on antibodies raised against TNF-alpha. For example, a mouse might be immunised against whole or a fragment of TNF-alpha, and an immunoglobulin library created which can be used in assays to screen for suitable binders to TNA-alpha, and suitable inhibitors of the TNA-alpha/TNF-alpha receptor interaction. The antibodies can be modified so as to be compatable with the subject e.g. a mouse antibody may be humanised. Such methods of raising, screening and humanising antibodies are well known in the art. Examples of antibody-based TNF-alpha inhibitors include Infliximab (Remicade®), Etanercept (Enbrel ®) and Adalimumab (Humira®).

As mentioned above, an inhibitor of TNF-alpha may be thalidomide (I) or a derivative thereof based on the thalidomide structure, such as like monothalidomides, dithiothalidomides, and trithiothalidomide.

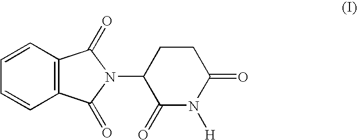

An inhibitor of TNF-alpha may also be a thalidomide analogue. Generally thalidomide analogues comprise two distinct classes of molecules. One class of compounds is the IMiDs (Immunomodulatory Imide Drugs), which consist of PDE4-inhibitors. The second class is called SelCiDs (Selective cytokine Inhibitory Drugs). The IMiDs are thought to be mechanistically similar to thalidomide. Both groups of compounds are potent TNF-alpha inhibitors (J. Blake Marriott et al. Cancer Research 2003; 63:593-599, 4. Laura G Corral, Gilla Kaplan. Ann Rheum Dis 1999; 1107-1113). Examples of IMiDs includes compounds of formulas (II), (III), (IV) and derivatives thereof. Examples of SelCiDs includes compounds of formulas (V), (VI), (VII) and derivatives thereof. Both classes of thalidomide analogue are within the scope of the present invention.

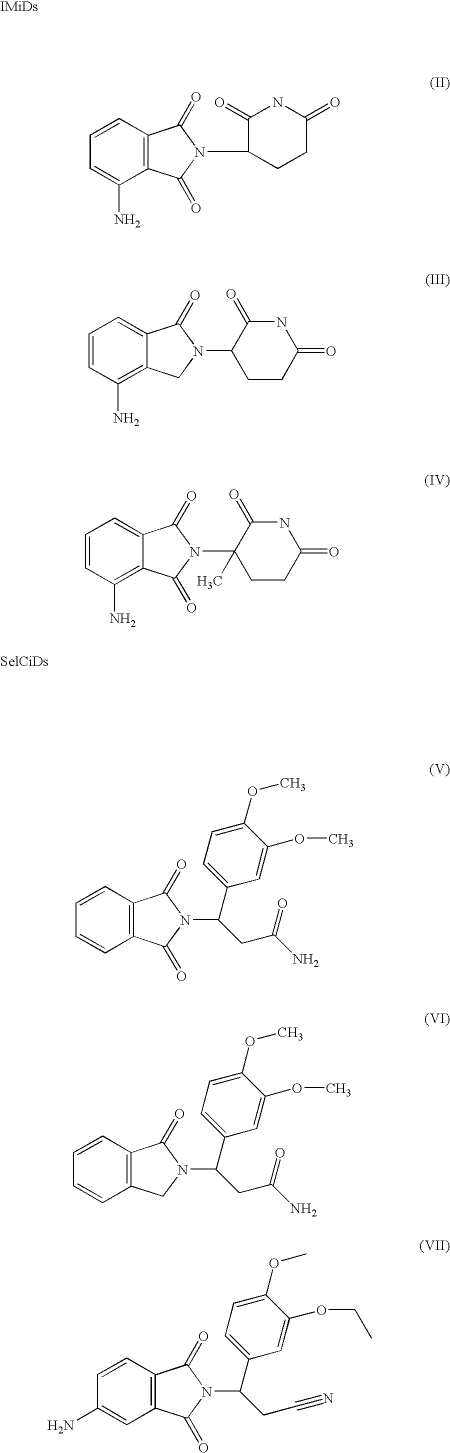

Inhibitors of TNF-alpha can be screened for use in the present invention by testing for their binding to TNF-alpha, their inhibition of the TNF-alpha/TNF-alpha receptor interaction, or their inhibition of TNF-alpha biosynthesis. Binding studies can be performed, for example, using microarray technology where a microarray (e.g. glass slide) is disposed with a plurality of locations of individual inhibitory compounds, and binding reactions are set up at each location. Reactions can be monitored using a variety of well known tools such as fluorescence, plasma resonance, ladiolabels, etc.

Individual and Combinations of Inhibitors

According to one aspect the invention, a composition comprises one or more inhibitors of TNF-alpha (e.g. thalidomide and infliximab). Owing to the properties of proliferating SMCs, the inventors find that a composition comprising combinations of inhibitors may also be effective at reducing a proliferating cell mass.

Derivatives

Stereoisomer, tautomers, racemates, prodrugs, metabolites, pharmaceutically acceptable salts, bases, esters, structurally related compounds or solvates of the TNF-alpha inhibitors are within the scope of the invention, unless otherwise stated.

The pharmaceutically acceptable salts of the inhibitors according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such a sarginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

The term “stereoisomer”, as used herein, defines all possible compounds made up of the same atoms bonded by the same sequence of bonds but having different three-dimensional structures which are not interchangeable, which the inhibitors of the present invention may possess. Unless otherwise mentioned or indicated, the chemical designation of an inhibitor herein encompasses the mixture of all possible stereochemically isomeric forms, which said compound may possess. Said mixture may contain all diastereomers and/or enantiomers of the basic molecular structure of said compound. All stereochemically isomeric forms of the inhibitors of the invention either in pure form or in admixture with each other are intended to fall within the scope of the present invention.

The inhibitors according to the invention may also exist in their tautomeric forms. Such forms, although not explicitly indicated in the inhibitors described herein, are intended to be included within the scope of the present invention.

For therapeutic use, the salts of the inhibitors according to the invention are those wherein the counter-ion is pharmaceutically or physiologically acceptable.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo.

Slow Release Formulation

Described above is the provision of a top coat for regulating the release of inhibitory compounds. Another aspect of the invention relates to a composition comprising additives which control inhibitor release. According to another embodiment of the invention, the composition is a slow release formulation. Accordingly, the stent may be provided with a large or concentrated dose of inhibitor. Once the stent is at the site of treatment, inhibitor is released at a rate determined by the formulation. This avoids the need for frequently replacing stents to maintain a particular dose. Another advantage of a slow release formulation is that the composition diffuses day and night, over several days or weeks.

One embodiment of the present invention is a stent comprising a composition as described herein, wherein said composition further comprises one or more slow release agents. Slow release agents may be natural or synthetic polymers, or reabsorbable systems such as magnesium alloys.

Among the synthetic polymers useful according to a slow release formulation of the invention are poly(glycolic) acid, poly(lactic acid) or in general glycolic- and lactic acid based polymers and copolymers. They also include poly caprolactones and in general, poly hydroxyl alkanoates (PHAs) (poly(hydroxy alcanoic acids)=all polyester). They also include Poly (ethylene glycol), poly vinyl alcohol, poly (orthoesters), poly (anhydrides), poly (carbonates), poly amides, poly imides, poly imines, poly (imino carbonates), poly (ethylene imines), polydioxanes, poly oxyethylene (poly ethylene oxide), poly (phosphazenes), poly sulphones, lipids, poly acrylic acids, poly methylmethacrylate (PMMA), poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass), siloxanes (Poly dimethyl siloxanes), hydroxyapatites, lactide-capronolactone, and any other synthetic polymer known to a person skilled in the art. Other synthetic polymers may be made from hydrogels based on activated polyethyleneglycols (PEGs) combined with alkaline hydrolyzed animal or vegetal proteins.

Among the natural derived polymers useful according to a slow release formulation of the invention, are poly aminoacids (natural and non natural), poly β-aminoesters. They also include poly (peptides) such as: albumines, alginates, cellulose/cellulose acetates, chitin/chitosan, collagene, fibrine/fibrinogen, gelatine, lignine. In general, proteine based polymers. Poly (lysine), poly (glutamate), poly (malonates), poly (hyaluronic acids). Poly nucleic acids, poly saccharides, poly (hydroxyalkanoates), poly isoprenoids, starch based polymers, and any other natural derived polymer known to a person skilled in the art.

Other polymers may be made from hydrogels based on activated polyethyleneglycols (PEGs) combined with alkaline hydrolyzed animal or vegetal proteins.

For both synthetic and natural polymers, the invention includes copolymers thereof are included as well, such as linear, branched, hyperbranched, dendrimers, crosslinked, functionalised (surface, functional groups, hydrophilic/hydrophobic).

The slow release composition may be formulated as liquids or semi-liquids, such as solutions, gels, hydrogels, suspensions, lattices, liposomes. Any suitable formulation known to the skilled man is within the scope the scope of the invention. According to an aspect of the invention, a composition is formulated such that the quantity of inhibitor is between less than 1% and 60 % of total slow-release polymer mass. According to an aspect of the invention, a composition is formulated such that the quantity of inhibitor is between 1% and 50%, 1% and 40%, 1% and 30%, 1% and 20%, 2% and 60%, 5% and 60%,10% and 60%, 20% and 60%, 30% and 60%, or 40% and 60% of total slow-release polymer mass.

Dose

The size of stent and concentration of composition thereon can be calculated using known techniques by the skilled person. The concentration of the composition is dependant on the potential of the compound to inhibit TNF-alpha per used microgram and might vary according to the activity of the inhibitor. According to one aspect of the invention, a stent is coated with a composition comprising TNF-alpha inhibitor such that the inhibitor concentration delivered to a subject is greater than or equal to 0.001, 0.005, 0.01, 0.05, 0.1, 0.5, 1, 5, 10, 20, 40, 60, 80, 100, 150, 200, 300, 400, 500, 600, 700, 800, 900, 1000 or mg inhibitor/mm² of stent, or a concentration in the range between any two of the aforementioned values.

The conventration of inhibitor per mm² of stent required to arrive at the above doses can be readily calculated by the skilled person.

Kit

A kit according to the invention may comprise at least one stent and separately, at least one composition of the present invention. The kit enables a technician or other person to coat a stent with a composition prior to insertion into a duct.

The composition, besides comprising at least one inhibitor of TNF-alpha, may contain additional substances that facilitate the coating of the stent by the end-user. The composition may contain, for example, fast evaporating solvents so as to allow the rapid drying of the stent. It may contain polymeric material to allow the inhibitors to adhere to the stent and facilitate its slow release.

The composition may be applied to the stent of the kit by any means known in the art. For example, by dipping the stent in the composition, by spraying the stent with the composition, by using electrostatic forces. Such methods are known in the art.

It is an aspect of the invention that the composition is provided in a container. For example, a vial, a sachet, a screw-cap bottle, a syringe, a non-resealable vessel, a resealable vessel. Such containers are any that are suitable for containing a composition and optionally facilitating the application of the composition to the stent. Indeed, some polymers to be used for the coating and the controlled release of the active compound, such as polyorthoesthers, are extremely unstable, are very sensitive to humidity and should be conserved in a cold atmosphere and in an argon atmosphere for instance. Some active products as well, such as rotenone, are sensitive to light and heat and should be preserved in dark and cold. In such a case, a container with the composition is kept separately from the bare stent. The interventional cardiologist may open the box containing the coating and apply it on the stent just before the intervention.

A kit may comprise more than one type of stent and more than one container of composition. A kit may provide a range of stent sizes, stent configurations, stents made from different materials. A kit may provide a range of vials containing different compositions with different inhibitors, different combinations of inhibitors, different combinations of polymers. A kit may facilitate the sequential application of more than one type of composition. A kit may contain instructions for use.

### SUMMARY OF SOME EMBODIMENTS OF THE INVENTION

One embodiment of the present invention is a medical stent provided with a composition comprising at least one inhibitor of TNF-alpha.

Another embodiment of the present invention is a stent as described above, wherein said stent is provided with one or more cavities configurated to contain and release said composition.

Another embodiment of the present invention is a stent as described above, wherein said stent is at least partly made from a material which is biodegradable in situ.

Another embodiment of the present invention is a stent as described above, wherein said stent comprises a magnesium based alloy.

Another embodiment of the present invention is a stent as described above, wherein said stent is at least partly made from a material which is non-biodegradable in situ.

Another embodiment of the present invention is a stent as described above, wherein said stent is at least partly provided with said composition.

Another embodiment of the present invention is a use of a composition comprising at least one inhibitor of TNF-alpha, for the preparation of a medicament for providing a medical stent for treating smooth muscle cell, SMC, proliferation.

Another embodiment of the present invention is a use as described above, wherein said stent is as defined above.

Another embodiment of the present invention is a kit comprising a) at least one medical stent and b) a composition comprising at least one inhibitor of TNF-alpha.

Another embodiment of the present invention is a kit as described above, wherein said stent is as defined above.

Another embodiment of the present invention is a medical stent, use or kit as described above, wherein said composition further comprises one or more slow release agents to facilitate slow release of inhibitor.

Another embodiment of the present invention is a medical stent, use or kit as described above, wherein said slow release agent is any of magnesium alloys, poly(glycolic) acid, poly(lactic acid) or in general glycolic- and lactic acid based polymers, copolymers, poly caprolactones and in general, poly hydroxyl alkanoate,s poly(hydroxy alcanoic acids), Poly (ethylene glycol), poly vinyl alcohol, poly (orthoesters), poly (anhydrides), poly (carbonates), poly amides, poly imides, poly imines, poly (imino carbonates), poly (ethylene imines), polydioxanes, poly oxyethylene (poly ethylene oxide), poly (phosphazenes), poly sulphones, lipids, poly acrylic acids, poly methylmethacrylate, poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass), siloxanes (Poly dimethyl siloxanes), hydroxyapatites, lactide-capronolactone, natural and non natural poly aminoacids, poly β-aminoesters, albumines, alginates, cellulose/cellulose acetates, chitin/chitosan, collagene, fibrine/fibrinogen, gelatine, lignine, proteine based polymers, Poly (lysine), poly (glutamate), poly (malonates), poly (hyaluronic acids), Poly nucleic acids, poly saccharides, poly (hydroxyalkanoates), poly isoprenoids, starch based polymers, copolymers thereof, linear, branched, hyperbranched, dendrimers, crosslinked, functionalised derivatives thereof, or hydrogels based on activated polyethyleneglycols combined with alkaline hydrolyzed animal or vegetal proteins.

Another embodiment of the present invention is a medical stent, use or kit as described above wherein said inhibitor of TNF-alpha is thalidomide.

Another embodiment of the present invention is a medical stent, use or kit as described above wherein said inhibitor of TNF-alpha belongs to the Immunomodulatory Imide Drug, IMiD, class of thalidomide analogues.

Another embodiment of the present invention is a medical stent, use or kit as described above, wherein said inhibitor is any compound of formula (II), (III) or (IV), or derivative thereof.

Another embodiment of the present invention is a medical stent, use or kit as described above, wherein said inhibitor of TNF-alpha belongs to the Selective cytokine Inhibitory Drugs, SelCiDs, class of thalidomide analogues.

Another embodiment of the present invention is a medical stent, use or kit as described above, wherein said inhibitor is any compound of formula (V), (VI) or (VII), or derivative thereof.

Another embodiment of the present invention is a medical stent, use or kit as described above, wherein said inhibitor of TNF-alpha is any of pentoxifylline, RWJ67657, EtOH (alcohol), Tyrphostin AG-556, AG126, AG128, s-adenosylmethionine, 5′-methylthioadenosine, inliximab (remicade), tgAAC94, entanercept (enbrel), D2E7 (humira), MP inhibitor G15402, TMI-1, SCIO-469, VX-745, BIRB 796, BB-2275, Uncaria tomentosa, SteiHap69, TACE inhibitors, ISIS25302, roflumilast, AS2868, AS2920, SPC-839, PEG-TNFRI, CDP-870, onercept, TIMP-3, DPC333, MMP inhibitors, Bortezomib (velcade), oxaspirodion, Shi-Bi-Lin, JM34, luteolin, urtica dioica, green tea polyphenols, tenidap, leflunimide (arava), curcuminoids, dexamethasone, vitamin D3, prostaglandin E2, or cyclosporin A.

Another embodiment of the present invention is a medical stent, use or kit as described above, suitable for use in inhibiting SMC proliferation.

Another embodiment of the present invention is a medical stent, use or kit as described above, wherein said SMC proliferation is restenosis or stenosis.

Another embodiment of the present invention is a medical stent, use or kit as described above, wherein the stent is placed in an artery or vein.

### EXAMPLES

The invention is illustrated by the following non-limiting examples. They illustrate the effectiveness of a selection of TNF-alpha inhibitors described above. The inhibitory properties of the other inhibitors not mentioned in the examples are known, and the skilled person may readily substitute the exemplified inhibitors with inhibitors such as listed above.

### Example 1

A composition comprising between 1 nanogram to 100 milligrams of thalidomide per square mm of undeployed stent and a suitable polymer is coated onto a balloon inflatable stent. The stent is introduced into a subject suffering from localised vascular stenosis using the percutaneous, transluminal, coronary angioplasty (PTCA) intervention. Six months after the intervention, an angiography is made of the area of the intervention. The degree of restenosis is calculated as a function of the percentage of patent vessel lumen.

### Example 2

3104 patients who successfully underwent a percutaneous coronary intervention (PCI) procedure were included in the GENetic Determinants of Restenosis (GENDER) project. GENDER was a prospective multicenter follow-up study. Systematic genotyping for six polymorphisms in the TNF gene was performed in order to demonstrate the role of six different TNF polymorphisms in the development of restenosis.

For the mouse study, ApoE*3-Leiden mice and TNF knockout mice were used to determine the impact of TNF-alpha on restenosis development after cuff placement around the femoral artery for 14 days. In another ApoE*3-Leiden mice group the cuffs were loaded with 1% (w/w) thalidomide, a TNF biosynthesis inhibitor.

Of the 3104 patients included, 304 patients had to undergo target vessel revascularisation (TVR). Patients with the −238A/A genotype and patients with the −1031C/C genotype needed TVR less frequently. The other TNF polymorphisms did not show a significant association with TVR. Twelve month TVR rates showed that patients with the haplotype −238G/−1031T had a higher risk for restenosis than the other types (p=0.02) Six month angiographic follow-up also showed an increased risk of restenosis for patients with this haplotype (p=0.002). A significant protective association was observed for the −238A allele (p=0.002)

In the mouse model arterial TNF mRNA was time-depently upregulated significantly. The TNF knockout mice as well as the mice treated with thalidomide showed significantly less neointima formation (p=0.014 and p=0.005 respectively)

The GENDER data show that TNF is involved in the development of restenosis in humans after PCI. This study demonstrated that patients with the TNF −238A/A and the −1031C/C genotypes have an decreased risk of restenosis. Huizinga et al. found that TNF production was lower for the −238A allele compared to the −238G allele (Huizinga T et. al. J. Neuroimmunol. 72:149-153, 1997.).

The mouse model demonstrated that mice that constitutively lack TNF show a reduction in neointima formation. Furthermore, the vascular levels of TNF protein were decreased when thalidomide was administered locally.

1. A medical stent provided with a composition comprising at least one inhibitor of TNF-alpha.

2. A medical stent according to claim 1, wherein said stent is provided with one or more cavities configured to contain and release said composition.

3. A medical stent according to claim 1, wherein said stent is at least partly made from a material which is biodegradable in situ.

4. A medical stent according to claim 1, wherein said stent comprises a magnesium based alloy.

5. A medical stent according to claim 1, wherein said stent is at least partly made from a material which is non-biodegradable in situ.

6. A medical stent according to claim 1, wherein said stent is at least partly provided with said composition.

7. A method for treating smooth muscle cell, SMC, proliferation comprising placing the medical stent of claim 1 on or adjacent to the proliferating SMC or in situ after removal of the proliferating SMCs.

8. (canceled)

9. A kit comprising a) at least one medical stent and b) a composition comprising at least one inhibitor of TNF-alpha.

10. A kit according to claim 9, wherein said stent is provided with one or more cavities configured to contain and release said composition.

11. A medical stent according to claim 1, wherein said composition further comprises one or more slow release agents to facilitate slow release of inhibitor.

12. A medical stent according to claim 11, wherein said slow release agent is any of magnesium alloys, poly(glycolic) acid, poly(lactic acid) or in general glycolic- and lactic acid based polymers, copolymers, poly-caprolactones and in general, poly hydroxyl alkanoates, poly(hydroxy-alcanoic acids), Poly-(ethylene glycol), poly-vinyl alcohol, poly (orthoesters), poly (anhydrides), poly (carbonates), poly-amides, poly-imides, poly-imines, poly (imino carbonates), poly (ethylene imines), polydioxanes, poly-oxyethylene (poly-ethylene oxide), poly (phosphazenes), poly-sulphones, lipids, poly-acrylic acids, poly-methylmethacrylate, poly-acryl amides, poly-acrylo nitriles (Poly-cyano acrylates), poly HEMA, poly-urethanes, poly olefins, poly-styrene, poly-terephthalates, poly-ethylenes, poly-propylenes, poly-ether ketones, poly-vinylchlorides, poly-fluorides, silicones, poly-silicates (bioactive glass), siloxanes (Poly dimethyl siloxanes), hydroxyapatites, lactide-capronolactone, natural and non natural poly aminoacids-, poly β-aminoesters, albumins, alginates, cellulose/cellulose acetates, chitin/chitosan, collagen, fibrin/fibrinogen, gelatin, lignin, protein based polymers, Poly (lysine), poly (glutamate), poly (malonates), poly (hyaluronic acids), Poly-nucleic acids, poly-saccharides, poly (hydroxyalkanoates), poly isoprenoids, starch based polymers, copolymers thereof, linear, branched, hyperbranched, dendrimers, crosslinked, functionalized derivatives thereof, or hydrogels based on activated polyethyleneglycols combined with alkaline hydrolyzed animal or vegetal proteins.

13. A medical stent according to claim 1, wherein said inhibitor of TNF-alpha is thalidomide.

14. A medical stent according to claim 1, wherein said inhibitor of TNF-alpha belongs to the Immunomodulatory Imide Drug, IMiD, class of thalidomide analogues.

15. A medical stent according to claim 14, wherein said inhibitor is any compound of formula (II), (III) or (IV), or derivative thereof

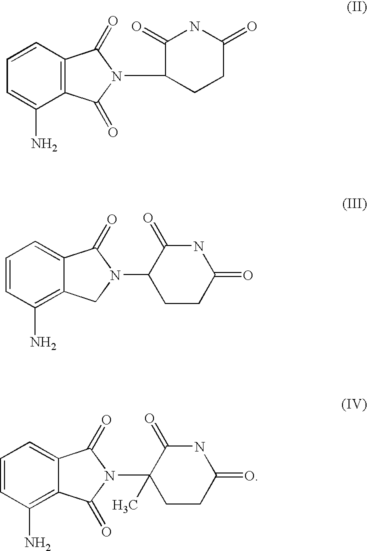

16. A medical stent according to claim 1, wherein said inhibitor of TNF-alpha belongs to the Selective cytokine Inhibitory Drugs, SeICiDs, class of thalidomide analogues.

17. A medical stent according to claim 16, wherein said inhibitor is any compound of formula (V), (VI) or (VII), or derivative thereof

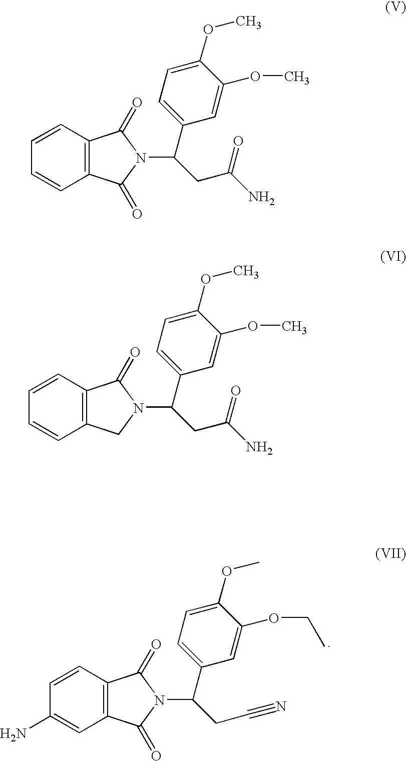

18. A medical stent according to claim 1, wherein said inhibitor of TNF-alpha is any of pentoxifylline, RWJ67657, EtOH (alcohol), Tyrphostin AG-556, AG126, AG128, s-adenosylmethionine, 5′-methylthioadenosine, inliximab (REMICADE), tgAAC94, entanercept (ENBREL), D2E7 (HUMIRA), MP inhibitor GI5402, TMI-1, SCIO-469, VX-745, BIRB 796, BB-2275, Uncaria tomentosa, SteiHap69, TACE inhibitors, ISIS25302, roflumilast, AS2868, AS2920, SPC-839, PEG-TNFRI, CDP-870, onercept, TIMP-3, DPC333, MMP inhibitors, Bortezomib (VELCADE), oxaspirodion, Shi-Bi-Lin, JM34, luteolin, urtica dioica, green tea polyphenols, tenidap, leflunimide (ARAVA), curcuminoids, dexamethasone, vitamin D3, prostaglandin E2, or cyclosporin A.

19. (canceled)

20. A method according to claim 7, wherein said SMC proliferation is restenosis or stenosis.

21. A method according to claim 7, wherein the stent is placed in an artery or vein.

22. A kit according to claim 9, wherein said composition further comprises one or more slow release agents to facilitate slow release of inhibitor.
